(19) United States
(12) Patent Application Publication
(10) Pub. No.: US 2025/0177784 A1
(43) Pub. Date: Jun. 5, 2025

(54) SYSTEM CONFIGURED TO AID IN TRAINING A PATIENT'S BREATHING IN PREPARATION FOR A RADIOTHERAPY TREATMENT

(71) Applicant: Brainlab AG, Munich (DE)

(72) Inventors: Kajetan BERLINGER, Munich (DE); Hagen KAISER, Munich (DE); Claus PROMBERGER, Munich (DE)

(21) Appl. No.: 18/846,005

(22) PCT Filed: Mar. 25, 2022
(86) PCT No.: PCT/EP2022/057976

§ 371 Date: Sep. 11, 2024

(51) Int. Cl. A61N 5/10

(52) U.S. Cl. CPC A61N 5/1068; A61N 5/1049

(57) ABSTRACT

Provided is a system and technique configured to aid in training a patient's breathing in preparation for a radiotherapy treatment. The system includes a mobile device configured to prompt, by an application running on the mobile device, the patient to perform a breathing exercise. The breathing exercise is directed to improving breath control. The mobile device is further configured to determine, by the application running on the mobile device and based on input data, one or more values indicating a training progress. The training of the patient's breathing includes one or more of: training the patient to create a breath pattern so as to match a predetermined breath pattern; training the patient to reproduce a prior breath pattern of the patient; and/or training breath-hold in preparation for breath-hold during radiotherapy treatment.

### FIELD OF THE INVENTION

The present invention relates to a system and computer-implemented method for training a patient's breathing in preparation for a radiotherapy treatment, a computer program product and a computer-readable medium.

### TECHNICAL BACKGROUND

Various radiotherapy applications require radiation of a chest area. For example, the treatment of breast cancers in most cases starts with a resection of the tumor, and is followed by radiotherapy. Clinical studies have shown that irradiating the tumor bed reduces the risk of recurrence dramatically. Negative side-effects may occur, as the heart—especially the RIVA “Ramus interventricularis anterior”, a coronary vessel—is very sensitive to radiation and thus the treatment may yield, in the long term, heart diseases. Especially regarding treatments of the left breast, the problem arises, because in this case the distance from target to the heart is comparatively short.

Therefore, most clinics plan and perform such radiotherapy treatments in the state of a deep inspiration breath-hold (DIBH). With inspiration the heart moves away from the area to be treated in inferior and posterior direction, and thus the heart is moved out of the radiation beam's path. Similarly to the heart, the liver moves significantly while breathing.

Other issues that occur in radiotherapy applications is that the tumor to be irradiated may move when the patient breathes. Since latency of signalling has to be taken into account, a prediction of the breathing pattern is required to accommodate for the latency. It is advantageous to have the patient breath as regularly and predictably as possible so as to correctly predict tumor position.

As an example, in many cases the patient is coached to perform a deep inspiration breath-hold during CT scanning. A treatment plan is created based on that CT scan. During treatment the patient is again coached to—as close as possible—reproduce the DIBH that was existent during CT scanning.

However, DIBH is difficult for a patient to perform, as it differs significantly from free breathing. In the course of the treatment, there is very little time to instruct the patient and the patient may also not be capable of performing this exerting type of breathing immediately. This limits the positive effects of DIBH for protecting the patient and/or predicting the tumor position taking into account breathing, thereby protecting the patient and also improving accuracy.

It is an objective of the present invention to better leverage DIBH for effectively protecting the patient and/or improve accuracy.

The present invention can be used for training a patient in preparation for radiotherapy treatment, e.g. in connection with a system for image-guided radiotherapy such as VERO® and ExacTrac®, both products of Brainlab AG.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The invention provides a system configured to aid in training a patient's breathing in preparation for a radiotherapy treatment, the system comprising a mobile device configured to prompt, by means of an application running on the mobile device, the patient to perform a breathing exercise, the exercise directed to improving breath control. The mobile device is configured to determine, by means of the application and based on input data, one or more values indicating a training progress. The training of a patient's breathing comprises training the patient to create a breath pattern so as to match a predetermined breath pattern and/or training the patient to reproduce a prior breath pattern of the patient and/or training breath-hold in preparation for breath-hold during radiotherapy treatment. The invention also provides a corresponding computer-implemented method, computer program product and computer-readable medium.

### GENERAL DESCRIPTION OF THE INVENTION

The invention provides a system configured to aid in training a patient's breathing in preparation for a radiotherapy treatment, the system comprising a mobile device configured to prompt, by means of an application running on the mobile device, the patient to perform a breathing exercise, the exercise directed to improving breath control. The mobile device is configured to determine, by means of the application and based on input data, one or more values indicating a training progress. The training of a patient's breathing comprises training the patient to create a breath pattern so as to match a predetermined breath pattern and/or training the patient to reproduce a prior breath pattern of the patient and/or training breath-hold in preparation for breath-hold during radiotherapy treatment.

The system of the present disclosure allows for the patient to make progress towards performing breathing in a controlled manner suitable for radiotherapy, e.g., DIBH breathing, Particularly, the patient can make progress successfully on their own, unsupervised, with technology readily available to them, and at a place and time that suits them. This allows for regular training and increases overall training effect. Even if the patient may not necessarily perform the breathing as used during radiotherapy, e.g., DIBH, as such during the training phase, they may learn to better control and/or hold their breath and/or increase awareness of their body when breathing.

The system of the present disclosure, thus, allows for supporting the patient and optionally also the clinical staff to better leverage DIBH for effectively protecting the patient and improving accuracy. It allows for a patient to train towards performing a stable, e.g., on the order of about 20 s, and reproducible DIBH. It may also allow for clinical staff to find out whether a patient is eligible for DIBH.

This can be seen as the clinic, e.g., instructions for breathing, can be brought to patient's home and vice versa.

The patient's breathing can be characterized by a breath pattern. A breath pattern may be characterized by a breath-hold time (also referred to in short as breath-hold) and/or a lift, e.g. of the chest, and/or regularity of breathing, among others. Breath-hold and/or lift are most important in the context of DIBH, as it requires that the chest lifts enough and can be held in the lifted position long enough.

Improving breath control may, for example, entail a patient being able to hold their breath for a longer period of time and/or achieving a higher lift, e.g., of the chest, and/or achieving a more constant breathing pattern and/or to be able to more easily and/or reliably replicate a breathing pattern.

Thus, one or more of the following training goals may be aimed at: stable breathing period, e.g. represented by a variation coefficient, stable breathing amplitude, e.g., represented by a variation coefficient, and stable breath rest position, optionally with a running average, e.g. represented by a standard deviation.

Values indicating a training progress may comprise past and present values of parameters representative of the patient's breath control, e.g., of their ability to create a breath pattern so as to match a predetermined breath pattern and/or their ability to reproduce a prior breath pattern and/or their ability to hold their breath. This may be a collection of prior and current values.

Input data and/or values indicating training progress may also be provided to a clinic for future use during the radiotherapy session, i.e., for clinicians to determine whether the patient is eligible for DIBH and/or to understand the expected capabilities and limitations of a patient. This will improve the planning of the treatment so as to increase reliability and reduce harm.

Input data may be any type of data that allows for deriving characteristics of the patient's breathing. This may include self-reported data on the patient's breathing. As an example, the mobile device may have a touch screen or be connected to a touch screen and the user interface may be displayed on the touch screen and configured to receive touch input.

Input data may also comprise previously stored data including general data, e.g., atlas data, and/or patient-specific data, e.g., medical data obtained in a clinical setting and/or historical data obtained by the application and/or patient input data. This is explained in more detail below.

As an example, the application may be configured such that the patient is instructed to press a button (start_inhale_event) and inhale as long possible. The timespan is measured and optionally shown. The patient is instructed to press a button when they need to exhale again (end_breath_hold_event). Alternatively or in addition, the events may be triggered automatically. Manual triggering might be error prone (particularly triggering the “end_breath_hold_event” manually). The internal sensors like gyroscope and accelerometer of the mobile device, e.g., a smartphone, could be used to automatically trigger the “end_breath_hold_event”. Alternatively or in addition, sensors like portable range scanners as found in AR googles, smartphones and gaming consoles could be used to measure chest movement of the patient and, based thereon, trigger “start_inhale” and “end_breath_hold” events automatically. Alternatively or in addition, range scanners like just described can be used to measure exactly how the chest is moving during the training and give information about the depth (quality) of the DIBH exercise and the reproducibility. As an example, a color coded distance to a stored reference surface may be augmented on the patient's body in real time.

According to the present disclosure, the mobile device may be configured to display an application user interface, the application user interface providing instructions guiding the patient through the breathing exercise and/or providing feedback regarding a current breathing exercise and/or a most recent breathing exercise and/or one or more past breathing exercises and/or providing feedback regarding the training progress and/or providing a user input interface for receiving user input. The application user interface may comprise at least a graphical user interface.

Guiding the patient through the breathing exercise may comprise that the mobile device, particularly the user interface, outputs instructions, e.g., audio, visual and/or haptic cues, instructing the patient on how to breathe for a given exercise. Guiding the patient through the breathing exercise may, in particular, comprise providing start, breath in, hold breath, breathe out, and/or stop instructions and/or instructions to increase or decrease lift of the chest and/or abdomen. The mobile device, particularly the user interface, may allow for outputting feedback, e.g. in the form of audio, visual and/or haptic cues, to the patient for the current exercise, e.g., a score or an indicator of successful completion, and/or may allow for a patient to review feedback for one or more previous exercises and/or to review training progress. The mobile device, in particular the application user interface, may also be configured to receive user input data, in particular, so as to allow for the patient to interact with the application. For example, a patient may input patient-specific data and/or exercise-specific data via the user interface and/or interact with the user interface so as to select a breathing exercise and/or interact with the user interface so as to access and view feedback or success for one or more current or previous exercises and/or training progress.

According to the present disclosure, the application user interface may be configured to provide immediate qualitative and/or quantitative feedback, in particular visual and/or acoustic and/or haptic feedback, regarding success of the current breathing exercise, optionally the user interface configured to directly or indirectly provide an instruction for increasing success, such as by visualizing a deviation of detected breathing behavior from a target breathing behavior, in particular to provide the instruction increasing success in addition to and/or as part of the feedback regarding the success of the current breathing exercise. As an example, providing the instruction increasing success may comprise instructions increase and/or decrease lift of the chest and/or abdomen.

In order to guide a patient through a breathing exercise, the user interface may visualize a goal to be reached, either in a manner that is directly related to the body of the patient, e.g. by visualizing how their body should be positioned and/or move during breathing and/or breath-hold and/or breath-relax, optionally overlaid with how their body is currently positioned and/or moving, e.g., as monitored via sensors. Alternatively or in addition, the goal might be visualized in a manner that is detached from the body of the patient, e.g., in a gamified or playful manner. As an example, arrows may be used to direct rise and fall of the chest, and a visual element may move upwards or downwards, in particular thereby collecting points and/or dodging obstacles.

Special gamified animations can be used to motivate the patient to not just train for breath-hold length but also depth. Conceivable animations comprise a bird who catches the worm, a snake that wants to bite into an apple, or the like.

In other words, the application may expose a gamified display (e.g., like gaming mobile applications) to train the patient in keeping the breath-hold. Gamification can include gathering points, showing progress, relevant animation of the procedure of inhaling.

As seen above, the feedback may be gamified or playful. The advantage of a gamified feedback is that instructions that are very technical will be difficult to follow and will not necessarily provide an understanding of success and possibilities to improve to a patient with limited medical knowledge. Thus, overall training success may be improved. A gamified display (e.g., goggles, screen) can motivate the patient to improve and train DIBH, thereby guarantee best treatment success.

The data indicative of success may be data that is, directly or indirectly, related to one of the training goals, for example related to at least one of a stability of breathing period, e.g., represented by a variation coefficient, a stability breathing amplitude, e.g., represented by a variation coefficient, and a stability of breath rest position, optionally with a running average, e.g. represented by a standard deviation, lift, e.g. height of lift, breath-hold time, precision of matching a breathing pattern, e.g., represented by a mean deviation from the breathing pattern.

According to the present disclosure, the application may be configured to prompt the mobile device to store data indicative of the success of the breathing exercise and/or to prompt the mobile device to access and optionally to visualize stored data indicative of the success of the breathing exercise, in particular wherein the data indicative of the success may be used for tracking success over time and/or may be included in the input data.

For example, storing the data may allow for recording and/or analyzing training progress and may also serve to allow for determining future breathing exercises based on the stored data indicative of the preceding training progress and optionally a previously defined goal. The stored data indicative of success, as described above, may also be included in the above-described input data, for example as part of historical data, which will be described in more detail below.

According to the present disclosure, the input data may comprise or be derived from sensor data, in particular sensor data obtained by one or more sensors comprised in the system, particularly comprised in the mobile device and/or connected to the mobile device, and/or received from one or more external sensors.

In other words, sensor data may be directly used as input data or data derived from sensor data may be used as input data, i.e., data obtained by a processing of sensor data. Such processing may comprise, for example, image processing and/or pattern recognition. Sensor data that is used as input data and/or from which input data is derived may be obtained by the system, particularly by sensors comprised in system. Alternatively or in addition, sensor data that is used as input data and/or from which input data is derived may be received by the system from external sensors, i.e., sensors that are not part of the system. The sensor data may be received directly from external sensors and/or via other entities external to the system. Examples for sensor data and data derived from sensor data will be described in detail further below.

The use of sensor data is advantageous, as it allows for objective evaluation of the patient's breathing, thereby improving the evaluation. In many cases, it is possible to achieve this improvement leveraging pre-existing functionalities, as mobile devices and various other devices like wearables or gaming devices already comprise sensors providing potentially suitable sensor data and/or data derived therefrom.

According to the present disclosure, the input data may comprise user input data obtained via a user interface of the mobile device, in particular user input data reporting completion of at least part of the breathing exercise.

In other words, input data may comprise self-reported data. An advantage of this is that the patient can perform breathing exercises in various environments and with various availability of equipment and have the data properly reflect completion of the exercises. For example, at one place, a patient may be equipped with various devices providing sensor data as input data for evaluating success. However, at other places, fewer devices may be available and/or their proper placement and/or operation may not be feasible. A user may then still perform the breathing exercise and self-report completion. Moreover, self-reported data may serve as supplementary data for sensor data, e.g., for times when no or insufficient sensor data is available and/or to provide a plausibility check and/or to set boundary conditions for evaluating sensor data.

As an example, user input data may be obtained via the user interface when a user interacts with a user interface element, for example a button that is part of the user interface. A user may confirm, via the user interface, that they completed a current breathing exercise and/or one or more previous breathing exercises. Alternatively or in addition, the user may indicate, via the user interface, the time of breathing in and/or the time of breathing out. From the time of breathing in and/or breathing out, success may be derived, e.g., based on the time between breathing in and breathing out.

According to the present disclosure, the input data may comprise usage information of the mobile device representative of an activity status of the application, in particular, times of activity and/or inactivity of the application. In other words, whether or not the application is actively used may be used as an indicator for success. This has similar advantages as the self-reported data, particularly in terms of supplementing other input data. However, use is made easier for the patient, as actively providing user input while also performing the breathing exercise may be difficult.

According to the present disclosure, the input data may comprise patient-specific data, in particular patient-specific physical data representing the patient's past and/or present physical characteristics. These characteristics may have an impact on the goals to be achieved in the breathing exercise and/or the evaluation of success and/or progress and/or may be used for processing sensor data. Thus, accuracy of the determining the training progress may be improved. Detailed examples of patient-specific data are provided further below. They may include at least one of weight, gender, size, pre-existing conditions, age, for example.

According to the present disclosure, the input data may comprise timer data, the timer data representative of a time of breathing in and/or a time of breathing out and/or a time elapsed between breathing in and breathing out. Such timer data may serve as an objective measure for determining success of a breathing exercise and/or training progress, e.g., on the basis of time of breath-hold. The timer for determining timer data may be triggered based on sensor data and/or based on a user input.

According to the present disclosure, the time of breathing in and/or the time of breathing out and/or the time elapsed between breathing in and breathing out may be derived from user input data reporting breathing in and/or breathing out. Thus, time of breath-hold can be self-reported, which has the advantages described above in the context of self-reported data. Self-reporting of times requires no skill or knowledge, thereby avoiding user errors and improving reliability of the self-reporting. As an example, the system may receive user input data starting and stopping a/the timer for determining the timer data described above. In another example, the system may automatically start the timer and signal the start of the timer, e.g., by outputting a visual, audio and/or haptic signal, and may receive user input data stopping the timer. The patient may provide or have a third party provide the user input starting and/or stopping the timer.

Alternatively or in addition, according to the present disclosure, the time of breathing in and/or the time of breathing out and/or the time elapsed between breathing in and breathing out may be derived from sensor data, particularly the sensor data described above. Thus, the time of breath-hold can be detected using sensors, which may be advantageous as it does not require the patient to focus on providing correct input, such that the patient may focus to a higher degree on the breathing exercise without an increased risk of errors that might be brought about by faulty user input when the patient's focus is shifted more to the breathing exercise. The system may be configured to process sensor-based determination of times and self-reported times, e.g., based on availability of sensor data and self-reported data at any given time. The advantages outlined above pertaining to the self-reported input data, particularly as supplement to sensor input data, apply similarly in this case.

The sensor data of the present disclosure, e.g., the sensor data mentioned above, may be configured to allow for deriving a state and/or activity of the patient, in particular, configured to allow for estimating a vital sign, such as heart rate or blood pressure, and/or a pose and/or movement of the patient. The sensor data may comprise current sensor data and/or sensor gathered by monitoring the patient over a period time.

Various examples of sensor data will be described below, which may be used alone or in combination as input data.

According to the present disclosure, the sensor data may comprise acceleration data representative of acceleration of one or more body parts, for example a torso, of the patient, in particular acceleration data obtained by means of one or more acceleration sensors comprised in the system, in particular, comprised in or connected to the mobile device, and/or acceleration data obtained by an external acceleration sensor. For example, one or more acceleration sensors, for example placed on the torso of a patient, may be configured to detect acceleration while the chest expands and contracts. Such sensor data may provide objective measures of the breathing of the patient. Acceleration sensors are often found in mobile devices, such that existing technology can be leveraged.

According to the present disclosure, the sensor data may comprise image data and/or video data depicting at least a torso of the patient, in particular image data and/or video data obtained by means of a camera and/or surface scanner comprised in the system, in particular, comprised in or connected to the mobile device, and/or obtained by an external camera and/or an external surface scanner. The system may be configured to derive expansion and/or contraction of the chest of the patient while breathing. To do so, image processing methods known in the art may be used, for example, based on detecting colors and/or shapes and/or landmarks of the surface of the patient in the image or video data. Image and/or video data may be obtained by a camera providing 2D image data and/or a camera or surface scanner providing 3D or depth image data, for example. The data from multiple cameras may also be combined to provide stereoscopic image data as sensor data. The use of image and/or video data allows for providing a wide variety of objective measures of the breathing of the patient. Moreover, recording image and/or video data is less error prone than other sensing methods may be, particularly when used inexpertly by a patient. In addition, image-processing technology is widespread, such that existing technology can be leveraged, e.g., with suitable adjustments to the application at hand.

According to the present disclosure, the sensor data may comprise pulse data representative of the patient's pulse, in particular pulse data obtained by means of a pulse sensing device comprised in the system, in particular, comprised in or connected to the mobile device, and/or obtained by an external pulse sensing device. The pulse slightly changes when breathing in and breathing out and particularly when performing specific breathing exercises, e.g., breath-hold. Accordingly, pulse data is an objective measure for the patient's breathing. It can be easily and reliably obtained by a wide variety of device and is not prone to error.

According to the present disclosure, the sensor data may comprise blood pressure data representative of the patient's blood pressure, in particular blood pressure data obtained by means of a blood pressure sensing device comprised in the system, in particular, comprised in or connected to the mobile device, and/or obtained by an external blood pressure sensing device. On the one hand, similar to pulse, blood pressure may be an objective measure of the patient's breathing and it can also be easily and reliably obtained. On the other hand, blood pressure data may also indicate that a certain breathing exercise causes an unexpected change in blood pressure, which may indicate, that the patient has not yet progressed as much as expected. Thereby, it can also serve as a direct measure of progress.

According to the present disclosure, the sensor data may comprise gyroscope data representative of patient movement and/or orientation, in particular gyroscope data obtained by means of a gyroscopic sensor comprised in the system, in particular, comprised in or connected to the mobile device, and/or obtained by an external gyroscopic sensor. For example, one or more gyroscopic sensors, for example placed on the torso of a patient, may be configured to detect chest expansion and contraction as the patient breathes. Such sensor data may provide objective measures of the breathing of the patient. Gyroscopic sensors are often found in mobile devices, such that existing technology can be leveraged.

As mentioned above, according to the present disclosure, the input data may comprise the patient specific data. The patient specific data may comprise age of the patient and/or weight of the patient. Alternatively or in addition, the patient specific data may comprise size and/or shape of the patient. Alternatively or in addition, the patient specific data may comprise pre-existing conditions of the patient. Alternatively or in addition, the patient specific data may comprise historical patient heart rate data representative of the patient's heart rate at one or more earlier times. Alternatively or in addition, the patient specific data may comprise historical patient blood pressure representative of the patient's blood pressure at one or more earlier times. Alternatively or in addition, the patient specific data may comprise historical patient breathing data characterizing the patient's breathing at one or more earlier times. Some patient specific data may be selected so as to supplement and/or assist in evaluating sensor data. For example, sensor data may be interpreted differently depending on the patient specific data. For example, historical data may allow for providing a baseline for sensor data. Shape and/or size may lead to different expectations in terms of what image data will depict when the patient breathes. Moreover, the progress may be evaluated differently for patients having different abilities and conditions. Thus, patient specific data may allow for more reliable evaluation of sensor data and/or improved evaluation of progress.

According to the present disclosure, the patient specific data may comprise data collected in the course of the patient using the application with and/or without supervision by a third party and/or data collected with supervision by a/the third party and including data obtained by devices external to the system. Data collected in the course of the patient using the application may comprise and/or be derived from any of the above-described input data, particularly user input data and sensor data. This data may be plentiful, and in many cases can be reliably collected without supervision, particularly in any environment including at home and traveling. Particularly, historical data, e.g., historical breathing data, may be derived from the prior use of the application. The data collected with supervision by the third party may comprise data obtained with supervision of a clinician and/or wherein the data obtained by devices external to the system may comprise data obtained by devices set up in a clinical setting. For example, this may include a patient's historical breathing data collected in a clinical setting, potentially using devices external to the system, and optionally collected when the patient performed breathing based on the instruction of a clinician. The devices external to the system may comprise one or more medical imaging devices, e.g., CT, MRI, or XRAY devices, and/or 3D surface imaging devices, like a 3D surface scanner and/or a depth camera. Such data may serve to study in more detail the patient's breathing, for example for use in evaluating subsequently obtained input data, particularly sensor data. Such data may also serve to define a baseline and/or a goal to be achieved by the breathing exercise and/or how to evaluate progress.

According to the present disclosure, the one or more sensors may comprise one or more first sensors comprised in the mobile device and configured to provide the acceleration data and/or image data and/or pulse data and/or blood pressure data and/or gyroscope data. Alternatively or in addition, according to the present disclosure, the one or more sensors may comprise one or more second sensors connected to the mobile device and configured to provide the acceleration data and/or image data and/or pulse data, and/or blood pressure data and/or gyroscope data, in particular, a camera and/or a surface scanner and/or a sensor comprised in a gaming system and/or a sensor comprised in wearable device. As an example, this may allow for leveraging widely available sensors or sensor systems used in devices targeted at end-users, which improves reliability and reduces the risk of misuse.

The invention also provides a computer-implemented method for training a patient's breathing in preparation for a radiotherapy treatment, the method comprising a mobile device prompting, by means of an application running on the mobile device, the patient to perform a breathing exercise, the exercise directed to improving breath control, and determining, by means of the application and based on input data, one or more values indicating a training progress, wherein training a patient's breathing comprises training the patient to create a breath pattern so as to match a predetermined breath pattern and/or training the patient to reproduce a prior breath pattern and/or training breath-hold in preparation for breath-hold during radiotherapy treatment. In particular, the input data may be input data as described above and/or claimed.

The method may comprise obtaining sensor data, particularly the sensor data as described above and/or claimed, by controlling one or more sensors, particularly the one or more sensors as described above and/or claimed, to obtain the sensor data, and/or by receiving external sensor data.

The method of the present disclosure may comprise the mobile device displaying an application user interface, the application user interface providing instructions guiding the patient through the breathing exercise and/or providing feedback regarding a current breathing exercise and/or a most recent breathing exercise and/or one or more past breathing exercises and/or providing feedback regarding training progress and/or providing a user input interface for receiving user input.

The method of the present disclosure may comprise the application user interface providing immediate qualitative and/or quantitative feedback, in particular visual and/or acoustic and/or haptic feedback, regarding success of the current breathing exercise, optionally the user interface directly or indirectly providing an instruction for increasing success, such as by visualizing a deviation of detected breathing behavior from a target breathing behavior, in particular providing the instruction increasing success in addition to and/or as part of the feedback regarding the success of the current breathing exercise.

The method of the present disclosure may comprise the application prompting the mobile device to store data indicative of the success of the breathing exercise and/or prompting the mobile device to access and optionally to visualize stored data indicative of the success of the breathing exercise, in particular wherein the data indicative of the success is used for tracking success over time and/or is included in the input data.

The present disclosure also provides a computer program product comprising instructions which, when the program is executed by a computer, cause the computer to carry out the method of the present disclosure.

The present disclosure also provides a computer-readable medium comprising instructions which, when executed by a computer, cause the computer to carry out the method of the present disclosure.

For example, the program, when running on at least one processor (for example, a processor) of at least one computer (for example, a computer) or when loaded into at least one memory (for example, a memory) of at least one computer (for example, a computer), causes the at least one computer to perform the above-described method. The invention may also relate to a (physical, for example electrical, for example technically generated) signal wave, for example a digital signal wave, carrying information which represents the program, for example the aforementioned program, which for example comprises code means which are adapted to perform any or all of the steps of the method according to the present disclosure. A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, for example the internet. The invention therefore may alternatively or additionally relate to a data stream representative of the aforementioned program.

The features and advantages outlined above in the context of the system similarly apply to the method, the computer program product, and the computer-readable medium described herein.

For example, the invention does not involve or in particular comprise or encompass an invasive step which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve or in particular comprise or encompass any surgical or therapeutic activity. The invention is instead directed as applicable to allowing for a user to learn breathing patterns and skills guided by the claimed system and method. For this reason alone, no surgical or therapeutic activity and in particular no surgical or therapeutic step is necessitated or implied by carrying out the invention.

### Definitions

In this section, definitions for specific terminology used in this disclosure are offered which also form part of the present disclosure.

### Computer Implemented Method

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

### Acquiring Data

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### Registering

The n-dimensional image of a body is registered when the spatial location of each point of an actual object within a space, for example a body part in an operating theatre, is assigned an image data point of an image (CT, MR, etc.) stored in a navigation system.

### Image Registration

Image registration is the process of transforming different sets of data into one co-ordinate system. The data can be multiple photographs and/or data from different sensors, different times or different viewpoints. It is used in computer vision, medical imaging and in compiling and analysing images and data from satellites. Registration is necessary in order to be able to compare or integrate the data obtained from these different measurements.

### Landmarks

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (for example on the surface of) a characteristic anatomical structure of the body part can also represent said structure. The landmark can represent the anatomical structure as a whole or only a point or part of it. A landmark can also for example lie on the anatomical structure, which is for example a prominent structure. An example of such an anatomical structure is the posterior aspect of the iliac crest. Another example of a landmark is one defined by the rim of the acetabulum, for instance by the centre of said rim. In another example, a landmark represents the bottom or deepest point of an acetabulum, which is derived from a multitude of detection points. Thus, one landmark can for example represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

### Atlas/Atlas Segmentation

Preferably, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a patient's body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image—the atlas data therefore contains or is comparable to medical image data. Such a comparison can be carried out for example by applying an image fusion algorithm which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects which together make up the complete structure. The atlas of a brain, for example, can comprise the telencephalon, the cerebellum, the diencephalon, the pons, the mesencephalon and the medulla as the objects which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

### Treatment Beam

The present invention relates to aid in training a patient's breathing in preparation for a radiotherapy treatment, which may involve the use of a treatment beam. The treatment beam treats body parts which are to be treated and which are referred to in the following as “treatment body parts”. These body parts are for example parts of a patient's body, i.e. anatomical body parts.

The present invention relates to the field of medicine and for example to the use of beams, such as radiation beams, to treat parts of a patient's body, which are therefore also referred to as treatment beams. A treatment beam treats body parts which are to be treated and which are referred to in the following as “treatment body parts”. These body parts are for example parts of a patient's body, i.e. anatomical body parts. Ionising radiation is for example used for the purpose of treatment. For example, the treatment beam comprises or consists of ionising radiation. The ionising radiation comprises or consists of particles (for example, sub-atomic particles or ions) or electromagnetic waves which are energetic enough to detach electrons from atoms or molecules and so ionise them. Examples of such ionising radiation include x-rays, high-energy particles (high-energy particle beams) and/or ionising radiation emitted from a radioactive element. The treatment radiation, for example the treatment beam, is for example used in radiation therapy or radiotherapy, such as in the field of oncology. For treating cancer in particular, parts of the body comprising a pathological structure or tissue such as a tumour are treated using ionising radiation. The tumour is then an example of a treatment body part.

The treatment beam is preferably controlled such that it passes through the treatment body part. However, the treatment beam can have a negative effect on body parts outside the treatment body part. These body parts are referred to here as “outside body parts”. Generally, a treatment beam has to pass through outside body parts in order to reach and so pass through the treatment body part.

Reference is also made in this respect to the following web pages: http://www.elekta.com/healthcare_us_elekta_vmat.php and http://www.varian.com/us/oncology/treatments/treatment_techniques/rapidarc.

### Arrangement of Treatment Beams

A treatment body part can be treated by one or more treatment beams issued from one or more directions at one or more times. The treatment by means of the at least one treatment beam thus follows a particular spatial and temporal pattern. The term “beam arrangement” is then used to cover the spatial and temporal features of the treatment by means of the at least one treatment beam. The beam arrangement is an arrangement of at least one treatment beam.

The “beam positions” describe the positions of the treatment beams of the beam arrangement. The arrangement of beam positions is referred to as the positional arrangement. A beam position is preferably defined by the beam direction and additional information which allows a specific location, for example in three-dimensional space, to be assigned to the treatment beam, for example information about its co-ordinates in a defined co-ordinate system. The specific location is a point, preferably a point on a straight line. This line is then referred to as a “beam line” and extends in the beam direction, for example along the central axis of the treatment beam. The defined co-ordinate system is preferably defined relative to the treatment device or relative to at least a part of the patient's body. The positional arrangement comprises and for example consists of at least one beam position, for example a discrete set of beam positions (for example, two or more different beam positions), or a continuous multiplicity (manifold) of beam positions.

For example, one or more treatment beams adopt(s) the treatment beam position(s) defined by the positional arrangement simultaneously or sequentially during treatment (for example sequentially if there is only one beam source to emit a treatment beam). If there are several beam sources, it is also possible for at least a subset of the beam positions to be adopted simultaneously by treatment beams during the treatment. For example, one or more subsets of the treatment beams can adopt the beam positions of the positional arrangement in accordance with a predefined sequence. A subset of treatment beams comprises one or more treatment beams. The complete set of treatment beams which comprises one or more treatment beams which adopt(s) all the beam positions defined by the positional arrangement is then the beam arrangement.

### Fixed (Relative) Position

A fixed position, which is also referred to as fixed relative position, in this document means that two objects which are in a fixed position have a relative position which does not change unless this change is explicitly and intentionally initiated. A fixed position is in particular given if a force or torque above a predetermined threshold has to be applied in order to change the position. This threshold might be 10 N or 10 Nm. In particular, the position of a sensor device remains fixed relative to a target while the target is registered or two targets are moved relative to each other. A fixed position can for example be achieved by rigidly attaching one object to another. The spatial location, which is a part of the position, can in particular be described just by a distance (between two objects) or just by the direction of a vector (which links two objects). The alignment, which is another part of the position, can in particular be described by just the relative angle of orientation (between the two objects).

### Medical Workflow

A medical workflow comprises a plurality of workflow steps performed during a medical treatment and/or a medical diagnosis. The workflow steps are typically, but not necessarily performed in a predetermined order. Each workflow step for example means a particular task, which might be a single action or a set of actions. Examples of workflow steps are capturing a medical image, positioning a patient, attaching a marker, performing a resection, moving a joint, placing an implant and the like.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates, schematically and not-to scale, a system according to the present disclosure;

FIG. 2 shows a mobile device and an exemplary user interface according to the present disclosure;

FIG. 3 shows a mobile device and an exemplary user interface according to the present disclosure;

FIG. 4 shows a mobile device and an exemplary user interface according to the present disclosure;

FIG. 5 shows a mobile device and an exemplary user interface according to the present disclosure;

FIG. 6 shows a mobile device and an exemplary user interface according to the present disclosure;

FIG. 7 is a schematic illustration of a supervised breathing setup;

FIG. 8 illustrates a method according to the present disclosure;

FIG. 9 illustrates another method according to the present disclosure; and

FIG. 10 illustrates the effect of proper breathing.

### DESCRIPTION OF EMBODIMENTS

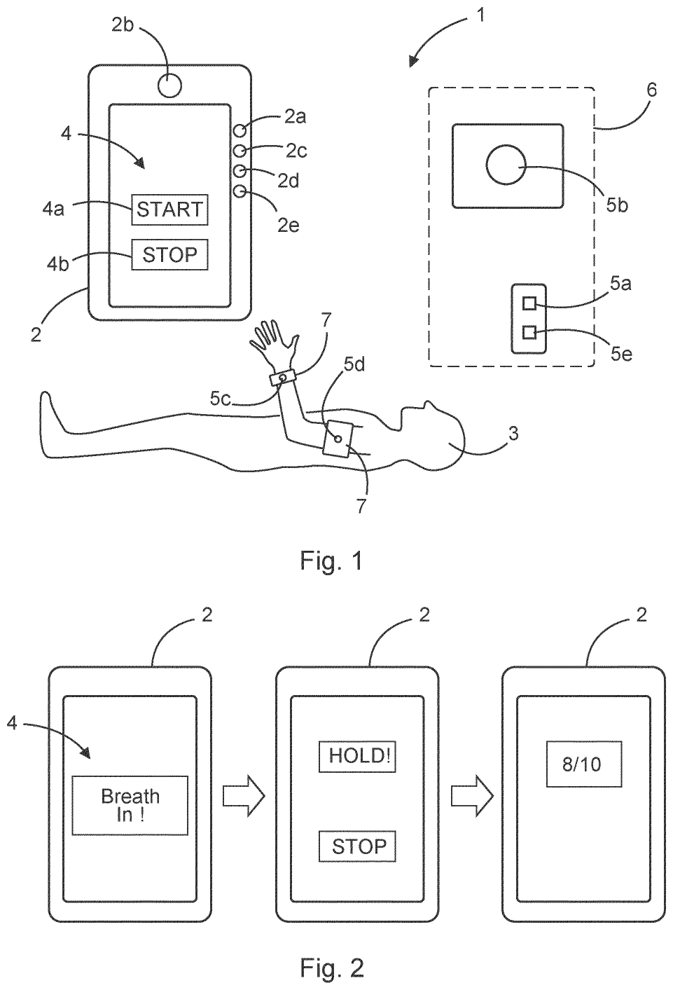

FIG. 1 illustrates a system 1 according to the present disclosure. The system comprises a mobile device 2 having a plurality of sensors 2a to 2e and a user interface 4, in this case graphical user interface, GUI, with GUI elements 4a and 4b. Moreover, in FIG. 1, optional sensors 5a to 5e of the system 1 are shown. In this example, the sensors 5a, 5b, and 5e are optionally comprised in a gaming system 6 and the sensor 5c or 5d may optionally be comprised in one or more wearable devices 7, for example a smartwatch.

For the sake of illustration, a patient, not part of the system, is also shown in FIG. 1 in a position lying down. The patient is also referred to as user hereinbelow.

The sensors may acceleration sensors 2a, 5a, a camera 2b, 5b and/or surface scanner, a pulse sensing device 2c, 5c, a blood pressure sensing device 2d, 5d and/or a gyroscopic sensor 2e, 5e.

In FIG. 2, an exemplary user interface 2 of the mobile device 2 is shown, wherein the user is instructed to breath in. Subsequently, the user is instructed to hold the breath. An interactive user interface element, here in the form of a “STOP” button may be provided that allows the user to input, e.g., by touching the display, when he breathed out. The user interface may then display a score as to success of the breathing exercise, e.g., in this example, the score “8/10”.

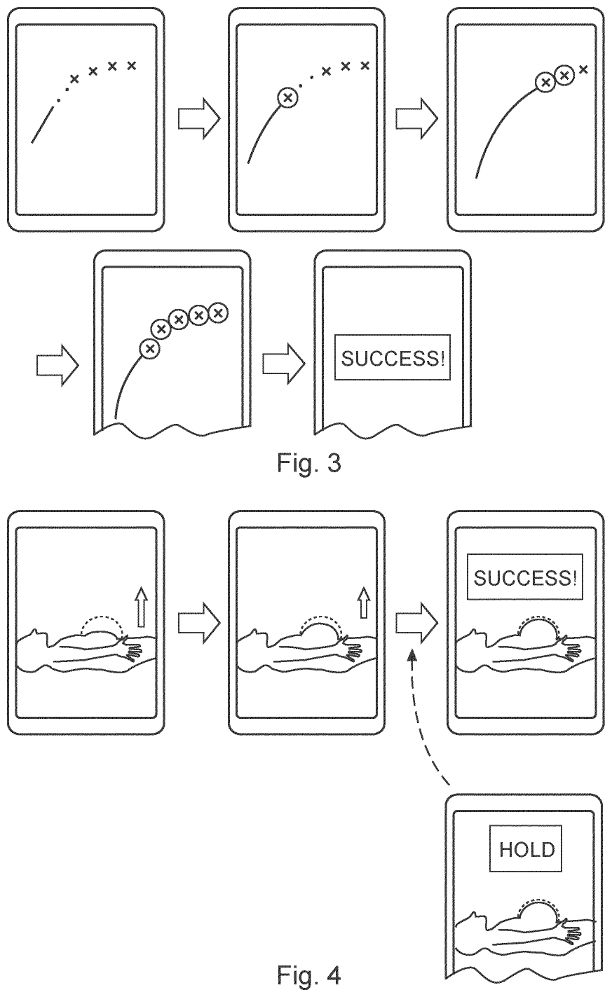

In FIG. 3, another exemplary user interface is shown. The user is shown a visualization that is representative of breathing in and holding breath over time. By correctly breathing in and holding their breath, the user can achieve the goals set, visualized here by a circle around each star. The user interface may inform the user of their success when they reach a certain number of stars.

In FIG. 4, another exemplary user interface is shown. Here, a human being is shown on the user interface and a target state is visualized by the dotted lines. The human being shown on the user interface may be a pre-defined graphic or represent image data of the user. An arrow indicates that the user's chest and stomach area should lift up to move towards the target, e.g., by breathing in deeper. When the user breathes, the shape of the human shown on the user interface changes in accordance with their breathing. Accordingly, the user can immediately derive whether they get closer to the target. For example, the chest and stomach of the human shown in the user interface may lift. When the target state is reached, the user interface may indicate success. Optionally, when the target state is reached and prior to indicating success, the user interface may instruct the user to hold their breath and, accordingly, target state.

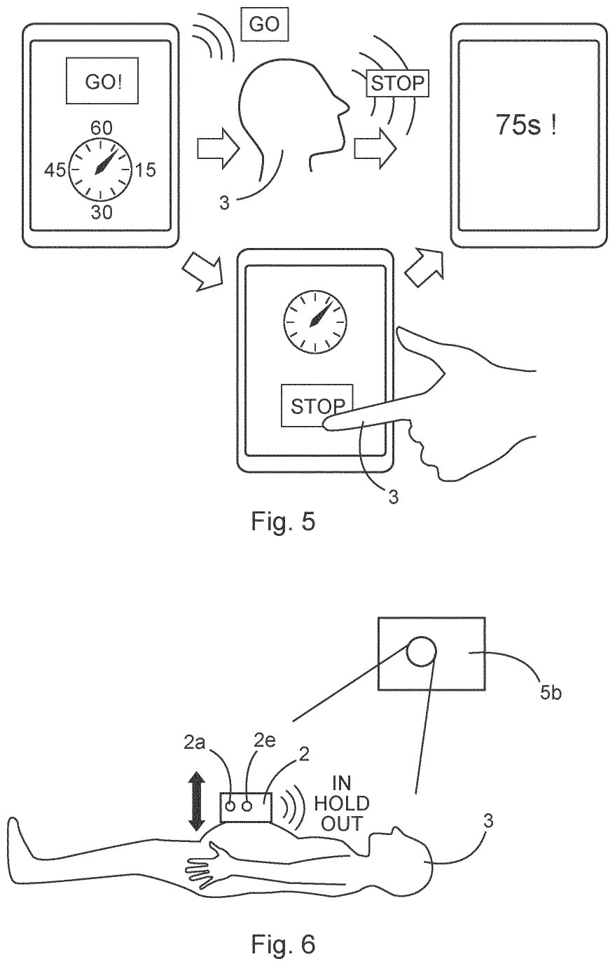

In FIG. 5, other exemplary user interfaces are shown. The user interface may instruct the user to start breathing in. An audio output may be used in addition or as an alternative to the visual instruction. Optionally, a watch may be displayed to indicate the time elapsed since start. The user may provide audio input or touch input to indicating breathing out. User interface may then, for example, display the time of breath-hold.

Various combinations of the above concepts and alternatives are conceivable.

FIG. 6 illustrates another system according to the present disclosure. A depth camera may monitor the user and provide image data as an input for automatically determining the user's breathing. Alternatively or in addition, the mobile device 2 having an acceleration and/or gyroscopic sensor may be placed on the user's abdomen and/or chest to provide acceleration and/or gyroscopic data as an input for automatically determining the user's breathing. Such a system is particularly suitable where it is difficult or impossible for a user to self-report, particularly for advanced breathing exercises, where a proper evaluation of success may require more than just a start and stop time.

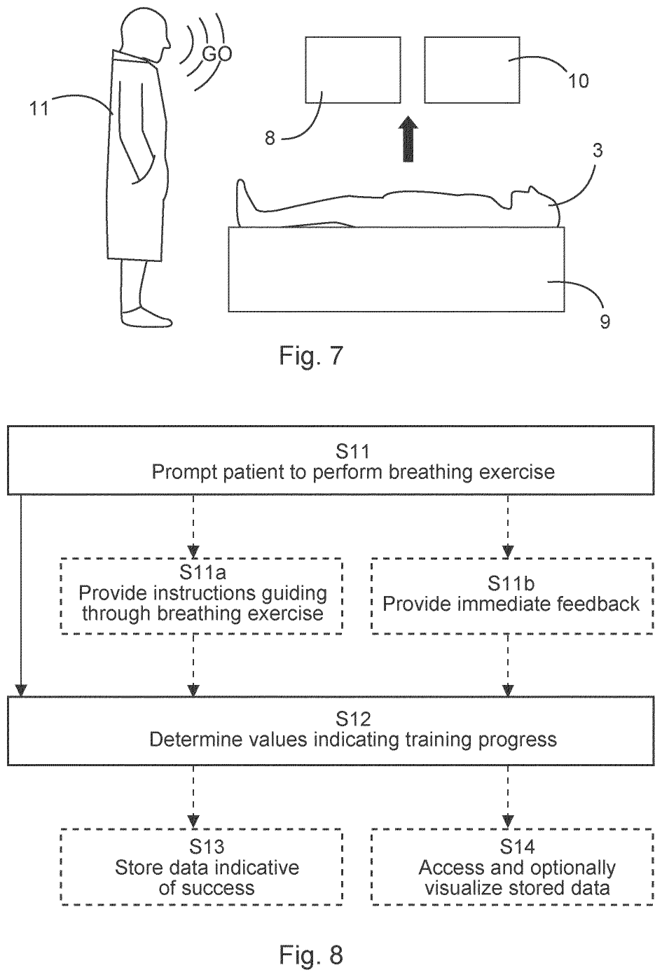

FIG. 7 illustrates a supervised breathing setup where a person 11 supervises the breathing exercises. This may be done in addition to, for example prior to or between, unsupervised breathing exercises. In this example, the supervising person may give various instructions, for example to start breathing in, to hold, or to breathe out. They may also use the mobile device or a different computing device to supervise and/or monitor the breathing. Alternatively or in addition to data from the mobile device, data from devices external to the system of the present disclosure may be used as input data for monitoring the breathing. As an example, a surface scanner 8 and/or medical imaging modalities 10 may be available as input data for supervising and/or monitoring the breathing. The user may be placed on a patient support 9 at the time of supervising and/or monitoring the breathing.

FIG. 8 illustrates a method for training a patient's breathing in preparation for a radiotherapy treatment according to the present disclosure, which may be performed using any system according to the present disclosure, particularly any of the systems described above or as claimed.

The method comprises a mobile device prompting in step S11, by means of an application running on the mobile device, the patient to perform a breathing exercise. The exercise is directed to improving breath control and may comprise exercises directed at free breathing, FB, and/or deep inspiration breath-hold, DIBH. The method comprises, in step S12, determining, by means of the application and based on input data, one or more values indicating a training progress. Training a patient's breathing comprises training the patient to create a breath pattern so as to match a predetermined breath pattern and/or training the patient to reproduce a prior breath pattern and/or training breath-hold in preparation for breath-hold during radiotherapy treatment.

The input data may be image data of the patient, acceleration and/or gyroscopic data from which movement due to breathing can be derived, pulse data, and/or blood pressure data. The input data may be input data obtained as described above, e.g., with one or more of sensors 2a to 2e and 5a to 5e. External data, e.g., external sensor data or patient-related data from a clinic, may also be received and used as input data.

As part of or subsequently to prompting the patient to perform a breathing exercise, the mobile device may optionally display, in step S11a, an application user interface, the application user interface may provide instructions guiding the patient through the breathing exercise and/or providing feedback regarding a current breathing exercise and/or a most recent breathing exercise and/or one or more past breathing exercises and/or providing feedback regarding training progress and/or providing a user input interface for receiving user input. The user interface may, for example, be a user interface as shown in any one of FIG. 2 to 5 or a combination thereof.

The method may also comprise, in step 11b, that the application user interface may optionally provide immediate qualitative and/or quantitative feedback, in particular visual and/or acoustic and/or haptic feedback, regarding success of the current breathing exercise, optionally the user interface directly or indirectly providing an instruction for increasing success, such as by visualizing a deviation of detected breathing behavior from a target breathing behavior, in particular providing the instruction increasing success in addition to and/or as part of the feedback regarding the success of the current breathing exercise.

Optionally, the application may prompt the mobile device to store data indicative of the success of the breathing exercise (step S13).

Optionally, the application may prompt the mobile device to access and optionally to visualize stored data indicative of the success of the breathing exercise (step S14).

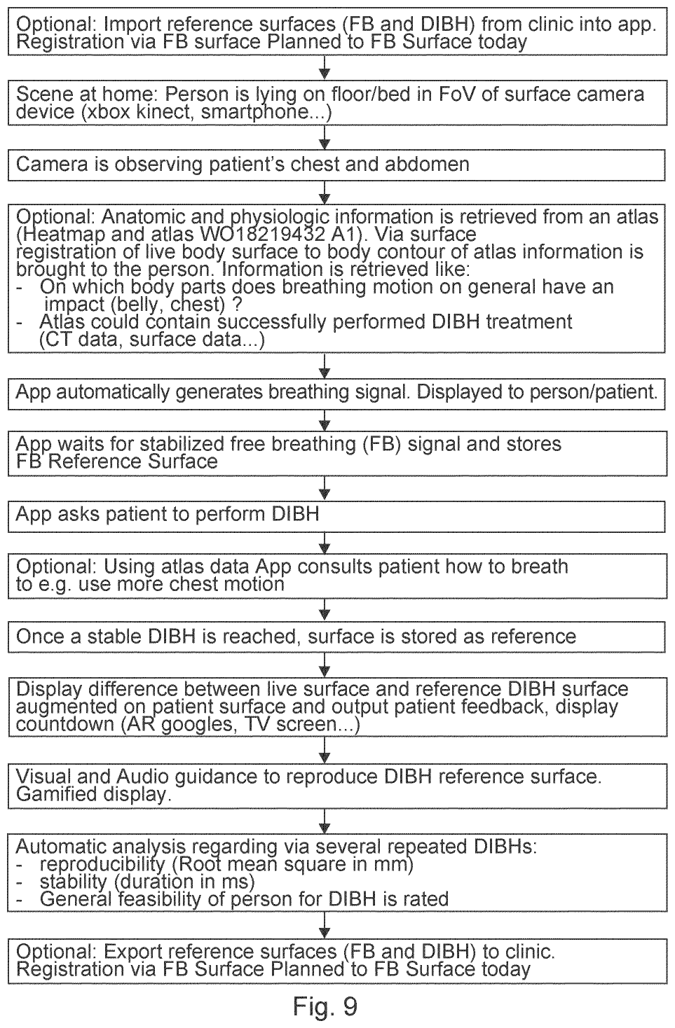

FIG. 9 illustrates another method according to the present disclosure, which may be performed using any system according to the present disclosure, particularly any of the systems described above or as claimed.

Optionally, the method may comprise importing reference surfaces (e.g., for free breathing, FB, and/or deep inspiration breath-hold, DIBH) from a clinic into an application. Registration may be performed, for example, via FB Surface Planned to FB Surface today.

The person, e.g. at home, is lying on a floor or bed in the field of view, FoV, of a surface camera device, which may be part of a gaming console, like the Xbox Kinect, or of the mobile device, which may be a smartphone.

The camera observes the patient's chest and abdomen.

Optionally, anatomic and physiologic information is retrieved from an atlas (cf., for example, WO18219432 A1). Via surface registration of live body surface to body contour of atlas information is brought to the person. The following information may be retrieved, for example:
On which body parts does breathing motion on general have an impact (belly, chest)?
Atlas data containing successfully performed DIBH treatment (CT data, surface data, or the like).

The application automatically generates a breathing signal. This may be displayed to person/patient via the user interface. Additional outputs, e.g., audio outputs are conceivable.

The application waits for stabilized free breathing (FB) signal and stores a FB Reference Surface.

The application instructs the patient to perform DIBH.

Optionally, using atlas data, the application may consult or instruction the patient how to breath, e.g., to use more chest motion.

Once a stable DIBH is reached, the surface is stored as reference.

A difference between live surface and reference DIBH surface augmented on patient surface may be displayed on the user interface and patient feedback may be output. Any output of the application may be performed via the mobile device or a device connected thereto, e.g., AR goggles or a TV screen. A countdown may be displayed. Visual and Audio guidance to reproduce DIBH reference surface. A gamified display may be provided.

An automatic analysis may be performed via several repeated DIBHs, the analysis regarding:
reproducibility (e.g., Root mean square in mm)
stability (duration in ms)
rating regarding general feasibility of person for DIBH

Optionally, reference surfaces may be exported (FB and DIBH), e.g. to a clinic. Registration via FB Surface Planned to FB Surface today may be performed.

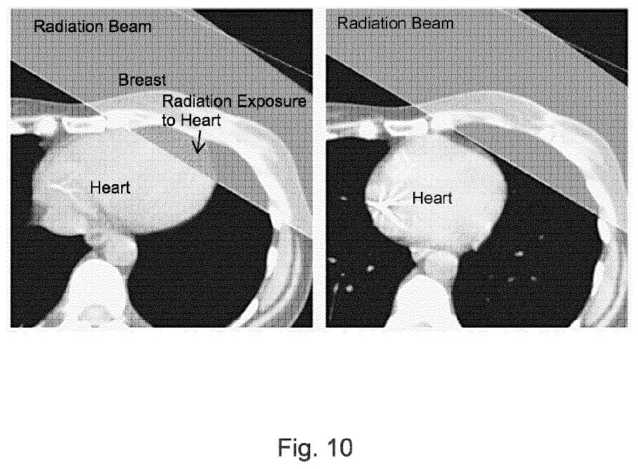

FIG. 10 illustrates the effect of proper breathing, specifically, how an organ like the heart can be brought outside of the range of a radiation beam by proper breathing.

The left image shows a beam path intersecting a patient's body in state of free breathing. The right image shows the same patient but now at state of a DIBH.

While the invention has been illustrated and described in detail in the drawings and foregoing description, such illustration and description are to be considered exemplary and not restrictive. The invention is not limited to the disclosed embodiments. In view of the foregoing description and drawings it will be evident to a person skilled in the art that various modifications may be made within the scope of the invention, as defined by the claims.

1. A system configured to aid in training a patient's breathing in preparation for a radiotherapy treatment, the system comprising:
a mobile device, wherein the mobile device is configured to:
prompt, by an application running on the mobile device, the patient to perform a breathing exercise, the breathing exercise directed to improving breath control; and
determine, by the application running on the mobile device and based on input data, one or more values indicating a training progress,
wherein training the patient's breathing comprises one or more of:
training the patient to create a breath pattern so as to match a predetermined breath pattern;
training the patient to reproduce a prior breath pattern of the patient and/or
training breath-hold in preparation for breath-hold during radiotherapy treatment.

2. The system of claim 1, wherein the mobile device is further configured to display an application user interface, the application user interface being configured to provide one or more of:
instructions guiding the patient through the breathing exercise;
feedback regarding one or more of: a current breathing exercise, a most recent breathing exercise, and/or one or more past breathing exercises;
feedback regarding the training progress; and/or
a user input interface for receiving user input.

3. The system of claim 2, wherein the application user interface is configured to provide immediate qualitative and/or quantitative feedback regarding success of the current breathing exercise and/or
wherein the application is configured to prompt the mobile device to
store data indicative of success of the breathing exercise; and/or
access stored data indicative of the success of the breathing exercise, wherein the data indicative of the success of the breathing exercise is used for tracking success over time and/or is included in the input data.

4. The system of claim 3, wherein the data indicative of the success of the breathing exercise is data related to one or more training goals, the data related to one or more training goals comprising data related to one or more of:
a stability of breathing period,
a stability breathing amplitude,
a stability of breath rest position,
a lift,
a breath-hold time, and/or
a precision of matching a breathing pattern.

5. The system of claim 1, wherein the input data comprises one or more of:
sensor data, wherein the sensor data is obtained via one or more of:
one or more sensors of the system; and/or
one or more external sensors;
user input data obtained via an application user interface of the mobile device, the user input data reporting completion of at least part of the breathing exercise;
usage information of the mobile device which is representative of an activity status of the application, the activity status representing at least one of times of activity of the application or times of inactivity of the application; and/or
patient-specific data, the patient-specific data representing at least one of the patient's past physical characteristics or present physical characteristics.

6. The system of claim 1, wherein the input data comprises timer data, the timer data being representative of one or more of:
a time of breathing in;
a time of breathing out; and/or
a time elapsed between breathing in and breathing out.

7. The system of claim 6, wherein one or more of the time of breathing in, the time of breathing out, and/or the time elapsed between breathing in and breathing out is derived from one or more of:
user input data reporting breathing in and/or breathing out; and/or
sensor data.

8. The system of claim 5, wherein the sensor data is configured to allow for deriving at least one of a state of the patient or activity of the patient, the sensor data being configured to allow for estimating one or more of:
a vital sign of the patient;
a pose of the patient; and/or
a movement of the patient.

9. The system of claim 5, wherein the sensor data comprises one or more of:
acceleration data representative of acceleration of one or more body parts of the patient, wherein the acceleration data is obtained via one or more of:
one or more acceleration sensors of the system; and/or
acceleration data obtained by an external acceleration sensor;
image data and/or video data depicting at least a torso of the patient, wherein the image data and/or video data is obtained via one or more of:
a camera;
a surface scanner of the system;
an external camera; and/or
an external surface scanner;
pulse data representative of a pulse of the patient, wherein the pulse data is obtained via one or more of:
a pulse sensing device of the system; and/or
an external pulse sensing device;
blood pressure data representative of a blood pressure of the patient, wherein the blood pressure data is obtained via one or more of:
a blood pressure sensing device of the system; and/or
an external blood pressure sensing device; and/or
gyroscope data representative of at least one of patient movement or patient orientation, wherein the gyroscope data is obtained via one or more of:
a gyroscopic sensor of the system; and/or
an external gyroscopic sensor.

10. The system of claim 5, wherein the input data comprises the patient-specific data and the patient-specific data comprises one or more of:
at least one of an age of the patient or a weight of the patient;
at least one of a or shape of the patient;
pre-existing conditions of the patient;
historical patient heart rate data representative of a heart rate of the patient at one or more earlier times;
historical patient blood pressure representative of a blood pressure of the patient at one or more earlier times; and/or
historical patient breathing data characterizing the patient's breathing at one or more earlier times.

11. The system of claim 10, wherein the patient-specific data comprises data collected in the course of the patient using the application according to one or more of:
with and/or without supervision by a third party; and/or
data collected with supervision by the third party and including data obtained by devices external to the system, wherein the data collected with supervision by the third party comprises data collected with supervision of a clinician wherein the data obtained by devices external to the system comprises data obtained by devices set up in a clinical setting.

12. The system of claim 9, the one or more sensors comprising:
one or more first sensors of the mobile device, the one or more first sensors configured to provide one or more of:
the acceleration data;
image data;
pulse data;
blood pressure data; and/or
gyroscope data;
one or more second sensors connected to the mobile device and configured to provide one or more of:
the acceleration data;
image data;
pulse data;
blood pressure data; and/or
gyroscope data;
the one or more sensors comprising one or more of:
a camera;
a surface scanner;
a sensor comprised in a gaming system, and/or
a sensor comprised in a wearable device.

13. A computer-implemented method for training a patient's breathing in preparation for a radiotherapy treatment, the method comprising:
prompting, by an application running on a mobile device, the patient to perform a breathing exercise, the breathing exercise directed to improving breath control;
determining, by the application running on the mobile device and based on input data, one or more values indicating a training progress, wherein training the patient's breathing comprises one or more of:
training the patient to create a breath pattern so as to match a predetermined breath pattern;
training the patient to reproduce a prior breath pattern; and/or
training breath-hold in preparation for breath-hold during radiotherapy treatment.

14. The method of claim 13, further comprising:
displaying, via the mobile device, an application user interface, the application user interface providing one or more of:
instructions guiding the patient through the breathing exercise;
feedback regarding one or more of:
a current breathing exercise;
a most recent breathing exercise; and/or
one or more past breathing exercises;
feedback regarding the training progress; and/or
a user input interface for receiving user input.

15. (canceled)

16. A non-transitory computer-readable storage medium storing a program comprising program instructions that, when executed on at least one processor of a computer of a mobile device or loaded onto the at least one processor of the computer of the mobile device, causes the computer to perform a method for training a patient's breathing in preparation for a radiotherapy treatment, comprising:
prompting, by an application executed on the at least one processor of the mobile device, the patient to perform a breathing exercise, the breathing exercise directed to improving breath control; and
determining, by the application executed on the at least one processor of the mobile device and based on input data, one or more values indicating a training progress, wherein training the patient's breathing comprises one or more of:
training the patient to create a breath pattern so as to match a predetermined breath pattern;
training the patient to reproduce a prior breath pattern; and/or
training breath-hold in preparation for breath-hold during radiotherapy treatment.

17. The method of claim 13, further comprising:
obtaining the input data by one or more of:
obtaining sensor data via one or more of:
one or more sensors of the mobile device and/or connected to the mobile device; and/or
one or more external sensors;
obtaining user input data via an application user interface of the mobile device, the user input data reporting completion of at least part of the breathing exercise;
obtaining usage information of the mobile device which is representative of an activity status of the application, the activity status representing at least one of times of activity of the application or times of inactivity of the application;
obtaining patient-specific data, the patient-specific data representing at least one of the patient's past physical characteristics or present physical characteristics; and/or
obtaining timer data, the timer data being representative of one or more of:
a time of breathing in;
a time of breathing out; and/or
a time elapsed between breathing in and breathing out.

18. The method of claim 17, wherein the obtaining the sensor data comprises one or more of:
controlling, via the application executed on the at least one processor of the mobile device, the one or more sensors of the mobile device and/or connected to the mobile device; and/or
receiving, by the application executed on the at least one processor of the mobile device, external sensor data from the one or more external sensors.

19. The method of claim 14, comprising:
providing, via the application user interface, immediate qualitative and/or quantitative feedback regarding success of the current breathing exercise; and/or
prompting, by the application running on the mobile device, the mobile device to:
store data indicative of success of the breathing exercise; and/or
access stored data indicative of the success of the breathing exercise, wherein the data indicative of the success of the breathing exercise is used for tracking success over time and/or is included in the input data.

20. The method of claim 19, comprising:
directly or indirectly providing, via the application user interface, an instruction for increasing success by visualizing a deviation of detected breathing behavior from a target breathing behavior; and/or
prompting, by the application running on the mobile device, the mobile device to:
visualize stored data indicative of the success of the breathing exercise.

21. The system of claim 3, wherein:
the application user interface is configured to directly or indirectly provide an instruction for increasing success by visualizing a deviation of detected breathing behavior from a target breathing behavior; and/or
the application is configured to prompt the mobile to:
visualize stored data indicative of the success of the breathing exercise.
